(19) United States
(12) Patent Application Publication
(10) Pub. No.: US 2010/0278935 A1
(43) Pub. Date: Nov. 4, 2010

(54) Immune System Modulator Formulation

(76) Inventor: William C. Stacey, Albemarle, NC (US)

Correspondence Address: THORPE NORTH & WESTERN, LLP., P.O. Box 1219, SANDY, UT, 84091-1219, US

(21) Appl. No.: 12/671,485

(22) PCT Filed: Jul. 30, 2008
(86) PCT No.: PCT/US08/71615

§ 371 Date: Jun. 28, 2010

Related U.S. Application Data
(60) Provisional application No. 60/962,624, filed on Jul. 30, 2007.

(51) Int. Cl. A61K 33/42; A61P 35/00; A61P 19/02; A61P 9/10; A61P 31/22; A61P 3/10

(52) U.S. Cl. 424/606

(57) ABSTRACT

Compositions and methods for treating and/or preventing a medical condition are provided. For example, in one aspect the present invention provides a composition for treating or preventing a medical condition in a subject. Such a composition may include a sodium phosphate compound, an ammonium compound, and a silicate compound. Such a composition may be formulated for a variety of administrations modes, including, without limitation, oral, intramuscular, intravenous, transdermal, buccal, etc.

### FIELD OF THE INVENTION

The present invention relates generally to therapeutic formulations and associated methods. Accordingly, the present invention involves the chemical and nutraceutical fields.

### BACKGROUND OF THE INVENTION

The immune system of mammals, particularly humans, is a complicated network of cells and biochemicals that act together to defend the body from pathogens. Such pathogens may include viruses, bacteria, protozoa, larger parasites, etc. The immune system may also protect the body against aberrant cell growth, as is seen with cancer. A weak immune system may fail to fight off the often destructive effects of such organisms, toxins, and/or poisons.

A strong immune system is high in Natural Killer (NK) cells that defend the body from foreign invaders. NK cells must be activated, however, in order to be effective. If the system responsible for activating these cells is deficient, NK cell activity decreases. Although many diseases that suppress NK cells are of unknown etiology, evidence has indicated that they may be treatable with various immune system modulation therapies.

The first line of defense against foreign pathogens is the barrier formed by the skin. If this barrier is penetrated, various cells in the body react to the presence of foreign matter. These cells include macrophages and neutrophils that engulf the foreign matter. These cells function without the need for specific recognition systems such as antibodies.

A second line of defense is the specific or adaptive immune system which may take days to respond to a primary invasion that has not previously been recognized by the body. In the specific immune system, antibodies are produced to allow recognition of specific foreign matter by the body. Once recognition has occurred, the body may react to and thus eliminate a particular invader.

As a result, methods and associated devices are being sought to provide modulation of the immune system to increase the body's defense capabilities.

### SUMMARY OF THE INVENTION

Accordingly, the present invention provides a composition for treating or preventing a medical condition in a subject. Such a composition may include a sodium phosphate compound, an ammonium compound, and a silicate compound. Such a composition may be formulated for a variety of administrations modes, including, without limitation, oral, intramuscular, intravenous, transdermal, buccal, etc.

Numerous silicate compounds are considered to be useful according to aspects of the present invention. In one aspect, for example, the silicate compound may include silicic acid, disilicic acid, trisilicic acid, metasilicic acid, orthosilicic acid, and combinations thereof. In one specific aspect, for example, the silicate compound may include a sodium salt. In another specific aspect the silicate compound may include sodium metasilicic acid. Additionally, any concentration of silicate compound in the composition that may be useful in treating or preventing a medical condition should be considered to be within the scope of the present invention. In one aspect, however, the silicate compound may be present at about 0.05 ppw to about 3.50 ppw in the composition. In another aspect, the silicate compound may be present at about 0.5 ppw to about 2.0 ppw in the composition. In yet another aspect, the silicate compound may be present at about 0.5 ppw to about 1.0 ppw in the composition.

Additionally, numerous ammonium compounds may be included in compositions according to aspects of the present invention. For example, in one aspect the ammonium compound may include ammonium chloride, ammonium hydroxide, and combinations thereof. In one specific example, the ammonium compound may include ammonium chloride. Additionally, any concentration of ammonium compound in the composition that may be useful in treating or preventing a medical condition should be considered to be within the scope of the present invention. In one aspect, however, the ammonium compound may be present at about 0.05 ppw to about 3.50 ppw in the composition. In another aspect, the ammonium compound may be present at about 0.75 ppw to about 2.5 ppw in the composition. In yet another aspect, the ammonium compound may be present at about 1.0 ppw to about 2.0 ppw in the composition.

Furthermore, a variety of sodium phosphate compounds are contemplated for use in the present invention. Examples of such compounds may include, without limitation, sodium phosphate dibasic anhydrous, sodium phosphate dibasic heptahydrate, sodium phosphate monobasic monohydrate, and combinations thereof. In one specific aspect, the sodium phosphate compound may include sodium phosphate dibasic anhydrous. Additionally, any concentration of sodium phosphate in the composition that may be useful in treating or preventing a medical condition should be considered to be within the scope of the present invention. In one aspect, however, the sodium phosphate compound may be present at about 0.05 ppw to about 3.50 ppw in the composition. In another aspect, the sodium phosphate compound may be present at about 0.75 ppw to about 2.5 ppw in the composition. In yet another aspect, the sodium phosphate compound may be present at about 1.0 ppw to about 2.0 ppw in the composition.

The present invention also provides methods for treating or preventing a medical condition. Such a method may include administering a composition to a subject to treat the medical condition, where the composition includes a sodium phosphate compound, an ammonium compound, and a silicate compound. Medical conditions that may be treated according to various aspects of the present invention may include any condition for which the composition exerts a therapeutic effect. In one aspect, however, the medical condition may include, without limitation, prostrate cancers, colorectal cancers, lung cancers, breast cancers, liver cancers, neural cancers, bone cancers, Human Immunodeficiency Syndrome, rheumatoid arthritis, multiple sclerosis, Epstein Barr Virus, fibromyalgia, chronic fatigue syndrome, diabetes, Bechets Syndrome, irritable bowel syndrome, Crohn's Disease, decubitus ulcers, trophic ulcers, immune system compromise due to radiation therapy or chemotherapy, superficial bruising, hematomas, and combinations thereof.

There has thus been outlined, rather broadly, various features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### DETAILED DESCRIPTION OF THE INVENTION

### Definitions

In describing and claiming the present invention, the following terminology will be used in accordance with the definitions set forth below.

The singular forms “a,” “an,” and, “the” include plural referents unless the context clearly dictates otherwise. Thus, for example, reference to “a compound” includes reference to one or more of such compounds, and reference to “the excipient” includes reference to one or more of such excipient.

As used herein, the terms “formulation” and “composition” may be used interchangeably and refer to a mixture of two or more compounds, elements, molecules, etc. In some aspects the terms “formulation” and “composition” may be used to refer to a mixture of one or more active ingredients with a carrier or other excipients.

As used herein, “active agent” and “active ingredient” may be used interchangeably to refer to a compound or substance that has measurable specified or selected physiologic activity when administered to a subject in a significant or effective amount.

As used herein, the terms “ppw” or “parts by weight” have reference to the amount of an ingredient present in a composition, whether the composition is a solid, solution, emulsion, powder, or any other form. Thus, the terms “ppw” or “parts by weight” may refer to the amount of one ingredient to one or more other ingredients to establish the relationship of such ingredients to each other, rather than to the whole of the composition. This term does not relate to the percent of each ingredient in the composition unless the total parts by weight is 100. In other words, the total “ppw” or “parts by weight” of a composition may be less than 100, or many times 100 depending on the amount of other ingredients such as excipients, blending agents, diluents, carriers, etc. Only when the total “ppw” or “parts by weight” of a composition is determined and the “ppw” or “parts by weight” of an ingredient is given can the percent of the ingredient in the total composition be ascertained. What is important is that the “ppw” or “parts by weight” of an ingredient in a total composition be sufficient to deliver an effective or desired amount of that ingredient in dosage form.

As used herein, “subject” refers to a mammal or fowl that may benefit from the administration of compositions or methods according to aspects of this invention. Examples of subjects include humans, and may also include other animals such as birds, horses, pigs, cattle, dogs, cats, rabbits, and aquatic mammals.

“Administration,” and “administering” refer to the manner in which an active agent is presented to a subject. Administration can be accomplished by various art-known routes such as oral, parenteral, transdermal, inhalation, implantation, etc.

The term “oral administration” represents any method of administration in which an active ingredient can be administered by swallowing, chewing, or sucking an oral dosage form. Such solid or liquid oral dosage forms are traditionally intended to substantially release and or deliver the active ingredient or ingredients in the gastrointestinal tract beyond the mouth and/or buccal cavity. Examples of solid dosage forms include conventional tablets, capsules, caplets, etc., which do not substantially release the drug in the mouth or in the oral cavity.

As used herein, “oral dosage form” refers to a formulation that is ready for administration to a subject through the oral route of administration. Examples of known oral dosage forms, include without limitation, tablets, capsules, caplets, powders, pellets, granules, liquids, gels, etc. In some aspects, powders, pellets, and granules may be coated with a suitable polymer or a conventional coating material to achieve, for example, greater stability in the gastrointestinal tract, or to achieve the desired rate of release. Moreover, capsules containing a powder, pellets or granules may be further coated. Liquids or gels may be packaged in capsules or other containers, or they may be swallowed as a liquid or gel.

The term “non-oral administration” represents any method of administration in which an active ingredient is not provided in a solid or liquid oral dosage form. It is appreciated that many oral liquid dosage forms such as solutions, suspensions, emulsions, etc., and some oral solid dosage forms may release some of the active ingredient in the mouth or in the oral cavity during the swallowing of these formulations. However, due to their very short transit time through the mouth and the oral cavities, the release of active ingredient from these formulations in the mouth or the oral cavity is considered de minimus or insubstantial. Thus, buccal patches, adhesive films, sublingual tablets, and lozenges that are designed to release the active ingredient in the mouth are non-oral compositions for the present purposes.

In addition, it is understood that the term “non-oral” includes parenteral, topical, transdermal, inhalation, implant, and vaginal or rectal formulations and administrations. Further, implant formulations are to be included in the term “non-oral,” regardless of the physical location of implantation. Particularly, implantation formulations are known which are specifically designed for implantation and retention in the gastrointestinal tract. Such implants are also considered to be non-oral delivery formulations, and therefore are encompassed by the term “non-oral.”

“Parenteral administration” can be achieved by injecting an active ingredient composition intravenously, intra-arterially, intramuscularly, intrathecally, subcutaneously, etc.

As used herein, “topical” refers to the route of administration whereby an active agent is applied to a body surface, such as skin or mucosal membranes. Topical administration includes, without limitation, transdermal forms of administration.

As used herein, “transdermal” refers to the route of administration taken by an active ingredient that is applied to and absorbed through an area of skin. In some aspects, the skin may be substantially unbroken. Thus the terms “transdermal formulation” and “transdermal composition” can be used interchangeably, and refer to formulations or compositions that are applied to a surface of the skin and transdermally absorbed. Examples of transdermal formulations include but are not limited to, ointments, creams, gels, transdermal patches, sprays, lotions, mousses, aerosols, nasal sprays, buccal and sublingual tablets and tapes or adhesives, vaginal rings, and pastes. The term “transdermal administration” thus refers to the transdermal application of a formulation or composition. Transdermal administration can be accomplished by applying, pasting, rolling, attaching, pouring, pressing, rubbing, etc., of a transdermal preparation or formulation onto a skin or mucosal surface of a subject. These and additional methods of administration are well-known in the art.

As used herein, “topical formulation” refers to a composition in which an active agent may be placed for direct application to a skin surface and from which an effective amount of the active ingredient is released.

The terms “skin,” “skin surface,” “derma,” “epidermis,” and similar terms are used interchangeably herein, and refer to not only the outer skin of a subject comprising the epidermis, but also to mucosal surfaces to which a composition may be administered. Examples of mucosal surfaces include the mucosa of the respiratory (including nasal and pulmonary), oral (mouth and buccal), vaginal, introital, labial, and rectal surfaces. Hence the terms “transdermal” encompasses “transmucosal” as well.

As used herein, “enhancement,” “penetration enhancement,” or “permeation enhancement,” refer to an increase in the permeability of the skin to an active ingredient, so as to increase the rate at which the active ingredient permeates through the skin. Thus, “permeation enhancer,” “penetration enhancer,” or simply “enhancer” refers to an agent, or mixture of agents that achieves such permeation enhancement. Several compounds have been investigated for use as penetration enhancers. See, for example, U.S. Pat. Nos. 5,601,839; 5,006,342; 4,973,468; 4,820,720; 4,006,218; 3,551,154; and 3,472,931. An index of permeation enhancers is disclosed by David W. Osborne and Jill J. Henke, in their publication entitled Skin Penetration Enhancers Cited in the Technical Literature, published in “Pharmaceutical Technology” (June 1998), which is incorporated by reference herein.

As used herein, an “effective amount” or a “therapeutically effective amount” of an active ingredient refers to a non-toxic, but sufficient amount of the drug, to achieve therapeutic results in treating a medical condition. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “pharmaceutically acceptable carrier” and “carrier” may be used interchangeably, and refer to any inert and pharmaceutically acceptable material that has substantially no biological activity, and makes up a substantial part of the formulation.

As used herein, “cluster designation” and “CD” can be used interchangeably, and refer to a well known classification system for antigens. A particular cluster designation number identifies an antigen that a “cluster” of antibodies bind to. As examples, CD2, CD3, CD4, CD5, CD7, and CD8 are often associated with antigens expressed on T-cells. CD 19, CD20, and CD22 are often associated with antigens expressed on B-cells. CD 16, CD56, and CD57 are often associated with antigens expressed on Natural-Killer cells. It should also be noted that the cluster designations may refer to antigens that are expressed on a variety of cell types. As one specific example, CD56 can be associated with antigens expressed on Natural Killer cells, hematopoietic cells, peripheral blood cells, tumor cells, spleen cells, leukemia cells, carcinoma cells, T-cells, liver, muscle cells, neuronal cells, lung cells, and myeloma cells.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free” of particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

The Invention

It has now been discovered that there is an increase in the body's natural immune system defenses following the administration of compositions according to aspects of the present invention. Following such administration, increases have been observed in various cells of the immune system that specifically attack, kill, and protect the body from infections from pathogens, such as, virus and bacteria.

The following theories are intended to provide a possible explanation for the results seen following administration of these compositions to a subject. It is thus not intended that the scope of the present invention be bound to these theories, and no limitations should be construed thereby.

Certain chemicals found on the surface of the T4 lymphocyte attract pathogens. The function of the T4 lymphocyte is to act as a control for the rest of the immune system. T4 cells direct and coordinate other cells of the immune system to respond to foreign matter. Such protection is accomplished by chemical signals passed from one cell to another or chemical signals released into the blood stream.

Once infected by a pathogen, the T4 cell no longer sends out the proper signals to stimulate other cells of the immune system. Many pathogens are attracted to the very receptors where the T4 cell receives messages from other cells. As the disease progresses, more and more T4 lymphocytes are infected and can no longer receive messages that communicate that the body is infected. Slowly the body's ability to fight off infection declines.

The compositions according to aspects of the present invention have been shown to increase cells that kill infected cells and cells that protect a subject's body from infection. This is accomplished by disrupting the attraction of a pathogen for the T4 lymphocyte receptor site. Pathogenic protein, (coded as non-self), is attracted to the receptor site on the T-cell (CD 4). Such a pathogen is held in place by a disulfide bond that renders the cell unable to see the interaction as a non-self protein interaction. Gone unchallenged for one cell cycle, the pathogen travels through the hapten on the receptor site into the T4 cell. If, on the other hand, the receptor site is cleared, proper pathogen signals are transmitted, thus causing CD 8 and CD 56 cells to recognize the non-self pathogen and to seek out and destroy the pathogen throughout the body. The compositions according to aspects of the present invention function to denature the disulfide bonds, thus denying the pathogen a hiding place. Once the T4 cell is free of the pathogen, it can once again send the proper signals to the other cells of the immune system. T4 cell populations decrease initially, but rebound quickly. CD 2 and CD 3 cell populations, for example, increase quickly, thus serving as an indication the pathogen is gone.

Accordingly, the present invention provides compositions and methods for treating or preventing a medical condition in a subject. In one aspect, for example, such a composition may include a sodium phosphate compound, an ammonium compound, and a silicate compound. It should be noted that the terms “sodium phosphate”, “ammonium,” and “silicate” also include all known forms of each compound, including freebases and salts.

Various silicate compounds are considered to be beneficial in the compositions according to aspects of the present invention. The term “silicate” may refer to silicic acid, disilicic acid, trisilicic acid, metasilicic acid, orthosilicic acid, and the like. Such definition also extends to freebase and salt forms of silicate compounds. Numerous salt forms are specifically contemplated, such as sodium salts, potassium salts, and the like. In one specific aspect, for example, the silicate compound may be a sodium salt. Sodium salt compounds can be useful as biologically compatible forms of many silicates. One such example is sodium metasilicic acid, or sodium metasilicate. Various forms of sodium metasilicic acid may be utilized, including various forms of hydration and non-hydration.

Silicic acid is a general name for a family of chemical compounds of silicon, hydrogen, and oxygen, with the general formula [SiOₓ(OH)₄₋₂ₓ]n. Examples include metasilicic acid (H₂SiO₃), orthosilicic acid (H₄SiO₄), disilicic acid (H₂Si₂O₅), and pyrosilicic acid (H6Si2O7). Silicic acids may be formed by acidification of silicate salts (such as sodium silicate) in aqueous solution.

The amount of a silicate compound utilized in compositions according to aspects of the present invention may vary widely depending on the intended use of the formulation, the nature and characteristics of the subject, as well as the nature of the condition to be treated. As such, any amount of a silicate compound may be utilized in the composition that produces a therapeutic effect in the subject. In one aspect, however, the silicate compound may be included at an amount of from about 0.05 ppw to about 3.50 ppw in the composition. In another aspect, the silicate compound may be included at an amount of from about 0.5 ppw to about 2.0 ppw in the composition. In yet another example, the silicate compound may be included at an amount of from about 0.5 ppw to about 1.0 ppw in the composition.

Various ammonium compounds may also be considered to be useful in the compositions according to aspects of the present invention. The ammonium ion is a positively charged polyatomic cation of the chemical formula NH₄⁺ that is formed by protonation of ammonia (NH₃). Ammonium is also a general name for positively charged or protonated substituted amines and quaternary ammonium cations N⁺R₄, where one or more hydrogen atoms are replaced by organic radical groups. It should be noted that the term ammonium may be used to refer to any substituted ammonium compound known that may be useful in the compositions according to aspects of the present invention. In one aspect, however, examples of such ammonium compounds may include, without limitation, ammonium chloride, ammonium hydroxide, and combinations thereof. In a more specific aspect, the ammonium compound may be ammonium chloride.

The amount of an ammonium compound utilized in compositions according to aspects of the present invention may vary widely depending on the intended use of the formulation, the nature and characteristics of the subject, as well as the nature of the condition to be treated. As such, any amount of an ammonium compound may be utilized in the composition that produces a therapeutic effect in the subject. In one aspect, however, the ammonium compound may be included at an amount of from about 0.05 ppw to about 3.50 ppw in the composition. In another aspect, the ammonium compound may be included at an amount of from about 0.75 ppw to about 2.5 ppw in the composition. In yet another example, the ammonium compound may be included at an amount of from about 1.0 ppw to about 2.0 ppw in the composition.

Various sodium phosphate compounds may also be considered to be useful in the compositions according to aspects of the present invention. Sodium phosphate is a sodium salt of phosphoric acid of the formula PO₄³⁻ in weakly acidic aqueous solutions. It should be noted that the term sodium phosphate may be used to refer to any sodium phosphate compound known that may be useful in the compositions according to aspects of the present invention. In one aspect, however, examples of such sodium phosphate compounds may include, without limitation, sodium phosphate dibasic anhydrous, sodium phosphate dibasic heptahydrate, sodium phosphate monobasic monohydrate, and combinations thereof. In a more specific aspect, the sodium phosphate compound may include sodium phosphate dibasic anhydrous.

The amount of a sodium phosphate compound utilized in compositions according to aspects of the present invention may vary widely depending on the intended use of the formulation, the nature and characteristics of the subject, as well as the nature of the condition to be treated. As such, any amount of a sodium phosphate compound may be utilized in the composition that produces a therapeutic effect in the subject. In one aspect, however, the sodium phosphate compound may be included at an amount of from about 0.05 ppw to about 3.50 ppw in the composition. In another aspect, the sodium phosphate compound may be included at an amount of from about 0.75 ppw to about 2.5 ppw in the composition. In yet another example, the sodium phosphate compound may be included at an amount of from about 1.0 ppw to about 2.0 ppw in the composition.

In one specific aspect of the present invention, a composition may include from about 0.05 ppw to about 3.50 ppw of sodium phosphate dibasic anhydrous, from about 0.05 ppw to about 3.50 ppw of sodium metasilicate non-hydrate crystalline, and from about 0.05 ppw to about 3.50 ppw of ammonium chloride granular in water. In another specific aspect of the present invention, a composition may include from about 0.75 ppw to about 2.5 ppw of sodium phosphate dibasic anhydrous, from about 0.5 ppw to about 2.0 ppw of sodium metasilicate non-hydrate crystalline, and from about 0.75 ppw to about 2.5 ppw of ammonium chloride granular in water. In yet another specific aspect of the present invention, a composition may include from about 1.0 ppw to about 2.0 ppw of sodium phosphate dibasic anhydrous, from about 0.5 ppw to about 1.0 ppw of sodium metasilicate non-hydrate crystalline, and from about 1.0 ppw to about 2.0 ppw of ammonium chloride granular in water. The concentration of each ingredient in water will obviously depend upon the amount or volume of water. In one aspect, a dose of such solution for administration will be formulated to have a titer such that an effective amount of the desired ingredient(s) will be present in such dosage form.

The compositions of the present invention may be formulated in numerous other ways, depending on the intended administration route for a particular composition. Such administrations routes may include, without limitation, oral administration, parenteral administration, transdermal administration, topical administration, etc. Oral administration may include any type of oral formulation, including tablets, capsules, liquids, gels, etc. In addition to swallowing, oral administration may include a variety of administration methods that by-pass the mouth into some portion of the gastrointestinal tract. Examples may include feeding tubes, gastrostomies, gastrointestinal implants, etc.

As has been described, parenteral administration may be by injection of the composition into the subject. Such injection may be, for example, intravenous, intraarterial, intramuscular, subcutaneous, intrathecal, intraperitoneal, or combinations thereof. Additionally, the composition may be non-invasively administered by a variety of topical methods, including transdermal delivery, transmucousal delivery, inhalational delivery, etc. Topical administration may include various methods of applying a topical formulation to the body, such as applications to the skin, mucosal formulations, inhalations, eye drops, ear drops, enemas, intranasal applications, vaginal applications, and the like.

Numerous excipients may be included in the compositions according to the various aspects of the present invention. An excipient is generally an inactive substance included in the composition. In many cases, active substances may not be easily administered and absorbed by the human body, and as such they need to be put in some appropriate form. Accordingly, the active substance may be dissolved or mixed with one or more excipients. Excipients can also be used to bulk up formulations containing very potent active ingredients, thus allowing for a more convenient and accurate dosage. Particular excipients may vary in a composition depending on the route of administration, the form of the active ingredients, etc. Examples of excipients may include, without limitation, carriers, stabilizers, antiadherents, binders, coatings, disintegrants, fillers, diluents, flavors, colors, sweeteners, glidants, lubricants, preservatives, sorbents, penetration enhancers, etc.

The present invention also provides methods for treating or preventing a medical condition. Such a method may include administering the compositions according to aspects of the present invention to a subject. Numerous medical conditions may be treated or prevented through the use of such compositions. Nonlimiting examples of such conditions may include prostrate cancers, colorectal cancers, lung cancers, breast cancers, liver cancers, neural cancers, bone cancers, Human Immunodeficiency Syndrome, rheumatoid arthritis, multiple sclerosis, Epstein Barr Virus, fibromyalgia, chronic fatigue syndrome, diabetes, Bechets Syndrome, irritable bowel syndrome, Crohn's Disease, periodontal disease, common cold, decubitus ulcers, trophic ulcers, immune system compromise due to radiation therapy or chemotherapy, superficial bruising, hematomas, and combinations thereof. In addition to treating medical conditions, formulations according to aspects of the present invention may be useful in enhancing a subject's level of energy.

It should be noted that dosing of the compositions according to aspects of the present invention may vary according to the medical condition being treated, the species of subject being treated, the physical makeup of the subject, etc. As such, the present invention should not be limited to a particular dosing regimen. As an example, however, a composition may be formulated having the concentrations as described herein, and may be administered to a subject as follows: from about 0.025 ml/kg to about 0.035 ml/kg daily for 4 consecutive days, followed by no administration for the next three days. Then from about 0.035 ml/kg to about 0.045 ml/kg daily for the next 4 consecutive days, followed by no administration for the next three days. Then from about 0.065 ml/kg to about 0.075 ml/kg daily for the next 4 consecutive days. This dosing schedule includes oral and parenteral types of administration. It will be realized that the relative proportion of each of the three required ingredients to each other will be within the parts by weight guidelines given above.

### EXAMPLES

A composition is prepared having 1.0 wt % of sodium metasilicate, 2 wt % sodium phosphate, and 2 wt % ammonium chloride granular in sterile water. An individual is given an oral administration of the composition according to the following schedule:

Days 1-4: 2 cc of the composition orally each day;

Days 5-7: no administration of the composition;

Days 8-11: 3 cc of the composition orally each day;

Days 12-14: no administration of the composition; and Days 15-18: 5 cc of the composition orally each day.

Following day 18, cluster designations CD 3, CD 4, CD 8, CD 19, and CD 56 are analyzed to verify the effectiveness of the treatment regimen.

Of course, it is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

1. A composition for treating or preventing a medical condition in a subject, comprising in a therapeutically effective amount:
a sodium phosphate compound;
an ammonium compound; and
a silicate compound.

2. The composition of claim 1, wherein the silicate compound may include a member selected from the group consisting of silicic acid, disilicic acid, trisilicic acid, metasilicic acid, orthosilicic acid, and freebases, salts, and combinations thereof.

3. The composition of claim 2, wherein the silicate compound is a sodium salt.

4. The composition of claim 3, wherein the silicate compound is sodium metasilicic acid.

5. The composition of claim 1, wherein the ammonium compound includes a member selected from the group consisting of ammonium chloride, ammonium hydroxide, and combinations thereof.

6. The composition of claim 5, wherein the ammonium compound is ammonium chloride.

7. The composition of claim 1, wherein the sodium phosphate compound includes a member selected from the group consisting of sodium phosphate dibasic anhydrous, sodium phosphate dibasic heptahydrate, sodium phosphate monobasic monohydrate, and combinations thereof.

8. The composition of claim 7, wherein the sodium phosphate compound is sodium phosphate dibasic anhydrous.

9. The composition of claim 1, wherein the sodium phosphate compound is present at about 0.05 ppw to about 3.50 ppw in the composition.

10. The composition of claim 1, wherein the sodium phosphate compound is present at about 0.75 ppw to about 2.5 ppw in the composition.

11. (canceled)

12. The composition of claim 1, wherein the silicate compound is present at about 0.05 ppw to about 3.50 ppw in the composition.

13. The composition of claim 1, wherein the silicate compound is present at about 0.5 ppw to about 2.0 ppw in the composition.

14. (canceled)

15. The composition of claim 1, wherein the ammonium compound is present at about 0.05 ppw to about 3.50 ppw in the composition.

16. The composition of claim 1, wherein the ammonium compound is present at about 0.75 ppw to about 2.5 ppw in the composition.

17-19. (canceled)

20. A method of treating or preventing a medical condition, comprising:
administering the composition of claim 1 to a subject.

21-27. (canceled)

28. The method of claim 20, wherein the composition is administered orally to the subject.

29. The method of claim 20, wherein the composition is administered by injection to the subject.

30. The method of claim 20, wherein the composition is administered intravenously to the subject.

31. The method of claim 20, wherein the medical condition may include a member selected from the group consisting of prostrate cancers, colorectal cancers, lung cancers, breast cancers, liver cancers, neural cancers, bone cancers, Human Immunodeficiency Syndrome, rheumatoid arthritis, multiple sclerosis, Epstein Barr Virus, fibromyalgia, chronic fatigue syndrome, diabetes, Bechets Syndrome, irritable bowel syndrome, Crohn's Disease, decubitus ulcers, trophic ulcers, immune system compromise due to radiation therapy or chemotherapy, superficial bruising, hematomas, and combinations thereof.
